SEQUENCE LISTING (1 sequence)

SEQ ID NO: 1 - protein, 272 aa, Artificial Sequence
  An organism  which includes naturally occurring mutants.
MGSSHHHHHHSSGLVPRGSHMFGFFKKDKAVEVEVPTQVPAHIGIIMDGNGRWAKKRMQPRVFGHKAGMEALQTVTKAANKLGVKVITVYAFSTENWTRPDQEVKFIMNLPVEFYDNYVPELHANNVKIQMIGETDRLPKQTFEALTKAEELTKNNTGLILNFALNYGGRAEITQALKLISQDVLDAKINPGDITEELIGNYLFTQHLPKDLRDPDLIIRTSGELRLSNFLPWQGAYSELYFTDTLWPDFDEAALQEAILAYNRRHRRFGGV

(19) United States
(12) Patent Application Publication
(10) Pub. No.: US 2007/0020743 A1
(43) Pub. Date: Jan. 25, 2007

(54) Undecaprenyl pyrophosphate synthase (upps)enzyme and methods of use

(76) Inventors: Nestor O. Concha, King of Prussia, PA (US); Cheryl A. Janson, King of Prussia, PA (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/498,281

(22) PCT Filed: Dec. 2, 2002
(86) PCT No.: PCT/US02/38715

§ 371 Date: Jun. 4, 2004

(30) Foreign Application Priority Data
Dec. 5, 2001 (US) 60337227

(51) Int. Cl. G06F 19/00; G01N 33/48; G01N 33/50; C12N 9/00

(52) U.S. Cl. 435/183; 702/19

(57) ABSTRACT

A novel Streptococcus pneumoniae UPPS native crystalline structure and a novel Streptococcus pneumoniae UPPS complex with the substrates FPP and IPP are identified.

### TECHNICAL FIELD OF THE INVENTION

The present invention relates generally to the identification of a novel undecaprenyl pyrophosphate synthase (herein “UPPS”) crystalline structure. In addition, it provides a novel undecaprenyl pyrophosphate synthase active site of a crystalline structure in complex with Isopentenyl pyrophosphate and in complex with farnesyl pyrophosphate and methods to use these crystalline forms and their active sites to identify and improve undecaprenyl pyrophosphate synthase inhibitor compounds, among other uses. These compounds are characterized by the ability to competitively inhibit binding of substrates or other like-molecules to the active site of UPPS.

### BACKGROUND OF THE INVENTION

Polyisoprenoid molecules constitute a diverse and essential group of cellular polymers that function as sugar transporters, pigments, vitamins, hormones, etc. These molecules are products of a condensation of isopentenyl units that are produced by either of two pathways, a mevalonate-dependent or a mevalonate-independent pathway. IPP is a building block in the synthesis of squalene from which steroids are produced, and it is also a precursor of geranyl pyrophosphate from which polyisoprenols are synthesized (C. K. Mathews, and K. E. van Holde, 1996, Biochemistry). The successive condensation reactions to produce polyisoprenols from IPP are catalyzed by prenyltransferases of which there are at least 16 different enzyme forms in four distinct classes. These classes differ in the stereochemistry of the reaction they catalyze, the chain length of the substrates they use, and the chain length of their products. In the proposed two-step general mechanism of prenyltransferases, the first step is the elimination of the diphosphate from the allylic substrate, followed by the attack of the incoming IPP substrate to form a new carbon-carbon bond, followed by a stereospecific removal of a proton and formation of a new double bond (K. Ogura, and T. Koyama (1998) Chemical Reviews 98, 1263-1276; S. Ohnuma, T. Koyama, and K. Ogura (1989) FEBS Letters 257, 71-74).

In one of their many functions throughout the cell, polyisoprenol molecules are used as essential sugar carriers in the biosynthesis of glycoproteins in mammalian cells, and as essential sugar carriers in the biosynthesis of the bacterial cell wall. The peptidoglycan of the bacterial cell wall of Gram positive bacteria is formed by alternating units of N-acetylglucosamine, NAcGlc and N-acetylmuramic acid, NacMur. A pentapeptide chain with sequence: (L-Ala)-(D-Glu)-(L-Lys)-(D-Ala)-(D-Ala) is linked through the N-terminal amino group of the peptide with the carboxyl group of the lactate moiety of NAcMur. These peptidoglycan chains are further cross-linked by connecting pentaglycine units. The NAcMur-pentapeptide is synthesized from UDP-NAcMur by successive additions of the corresponding amino acids catalyzed by various ligases, after which the NacMur-pentapeptide is transferred to undecaprenolphosphate with the release of UMP. While linked to the undecaprenol carrier, NAcGlc and five glycine residues are added from Gly-tRNA. Subsequently, the NacGlc-NacMur-pentapeptide-pentaglycine intermediate is transferred to a peptidoglycan acceptor with the release of undecaprenyl pyrophosphate. In the next step, the terminal D-Ala is cleaved and released as adjacent peptidoglycan chains are cross-linked between the penultimate D-Ala of the first chain and the •—NH group of the lysine in the second chain. It is noteworthy that several molecules with antibacterial activity block various steps along this pathway. For example, bacitracin blocks the hydrolysis of the phosphodiester bond of undacaprenyl pyrophosphate to produce undecaprenylphosphate; vancomycin blocks the transfer of NacGlc-NacMur-pentapeptide-pentaglycine to the acceptor; and penicillin and cephalosporins block the transpeptidation, or cross-linking, between adjacent peptidoglycan chains.

The sugar-carrier function of undecaprenolphosphate in bacteria is performed in mammalian cells by dolicholphosphate during N-linked glycosylation of peptides in the ER. The human and bacterial homologous enzymes share about 34% amino acid sequence identity. Unlike undecaprenol, formed by 11 isopentenyl units, the dolichol molecule is nearly twice as large with 19-21 units. The amino acid sequence alignment of related UPPSs shows that contrary to other prenyl transferases, UPPS does not have the DDXXD sequence motif (A. Chen, P. A. Kroon, and C. D. Poulter (1994) Protein Science 3, 600-607). Instead, several stretches of highly conserved residues are localized around a shallow cleft on the surface of the protein around the active site. This observation is consistent with the topology observed in the crystal structure of UPPS from Micrococcus luteus B-P 26, (M. Fujihashi, N. Shimizu, Y-W. Zhang, T. Koyama, and K. Miki (1999) Acta Crystallographica D55, 1606-1607; M. Fujihashi, Y-W. Zhang, Y. Higuchi, X-Y. Li, T. Koyama, and K. Miki (2001) Proceedings of the National Academy of Sciences, U.S.A. 98, 4337-4342).

UPP is synthesized by the consecutive action of two enzymes: FPPS and UPPS. The crystal structure of FPPS in complex with FPP shows the FPP molecule bound in a deep pocket at a domain interface with a magnesium ion coordinated between the pyrophosphate and two aspartate residues in the DDXXD motif characteristic of these class of farnesyltransferases (L. C. Tarshis, M. Ynag, C. D. Poulter, and J. C. Sachettini (1994) Biochemistry 33, 10871-10877). The FPPS product is an (all-E)-farnesyl diphosphate, one of the substrates for UPPS. UPPS is a (Z)-polyprenyldiphosphate synthase (prenyltransferase type IV) that catalyzes the sequential Z-addition of eight IPP molecules to an all-E-FPP to produce a E,Z-mixed-C₅₅-isoprenyldiphosphate product, undecaprenyl pyrophosphate (M. Ito, M. Kobayashi, T. Koyama, and K. Ogura (1987) Biochemistry 26, 4745-4750): 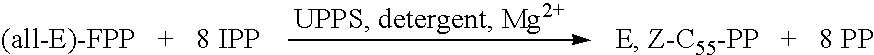
The S. pneumomiae UPPS has a ^{FPP}Kₘ of 0.5 μM and a ^{IPP}Kₘ of 3.6 μM with a pH optimum of 7.5-8.0. The monomer has a pI of 5.1, a Mr•29,000 Da and is physiologically and catalytically active as a dimer. UPPS is an essential enzyme present in both Gram-positive and Gram-negative pathogens, except in Mycoplasma (J. D. Mutle, and C. M. Allen (1989) Archives in Biochemistry and Biophysics 230, 49-60; I. Takahashi, and K. Ogura (1982) Journal of Biochemistry 92, 1527-1537; N. Shimizu, T. Kagawa, and K. Ogura (1998) Journal of Biological Chemistry 273, 19476-19481; C. M. Apfel, B. Takaca, M. Fountoulakis, M. Stieger, and W. Keck (1999) Journal of Bacteriology 181, 483-492).

The present invention provides a crystal structure of UPPS from Streptococcus pneumoniae in its native state, and in complex with the substrates FPP and IPP. The structures show that UPPS is a dimer with an extensive contact area along a dimer interface. A shallow cleft harbors numerous conserved residues and delimits an active site. Several of these residues are disordered in a native enzyme but become well ordered in substrate-bound complexes. The crystal structures of the complexes with each of two substrates, FPP and IPP, provide a detailed description of these substrates' mode of binding, a structure of the Michaelis complex, certain critical residues involved in binding of substrates. The bound substrates and the residues that appear responsible for catalysis indicate a reaction mechanism, and map the available binding pockets used by inhibitors.

### SUMMARY OF THE INVENTION

In one aspect, the invention provides a composition comprising a UPPS in crystalline form.

In another aspect, the present invention relates to a UPPS protein that is derived from Streptococcus pneumoniae comprising the amino acid sequence shown in SEQ ID No. 1 having coordinates of any or all of Tables I-III, said protein in an essentially pure native form, or a homolog thereof. In a preferred embodiment, the invention provides a UPPS composition wherein said UPPS is a dimer.

In another aspect, the present invention provides a crystalline form of Streptococcus pneumoniae UPPS as derived from models of UPPS comprising coordinates of any or all of Tables I-III.

In yet another aspect, the invention provides a UPPS protein in crystalline form having coordinates of Table IA, and interatomic distances and angles of active site residues listed in Table IIA and/or Table IIIA, respectively, in an essentially pure native form or a homolog thereof.

In yet another aspect, the invention provides a prenyltransferase of a Streptococcus pneumoniae UPPS in its native crystalline form. A preferred embodiment of the invention provides a prenyltransferase wherein said prenyltransferase has an active site formed by the amino acids Arg247, Gly250, Arg206, Arg200, Ser208, Tyr217, Asp28, Tyr70, Ile26, Phe72, Asn76, Met27, Ala71, particularly as ligands to IPP.

In yet another aspect, the invention provides a prenyltransferase wherein said prenyltransferase has an active site formed by the amino acids Asp28-Arg32, Arg79, Met27, His45, Gly48, Met49, Leu52, Ala71, Tyr70, Leu90, Pro91, Phe94, Phe149, particularly as ligands to FPP.

In another aspect, the invention provides a composition comprising a prenyltransferase in complex with FPP as characterized by the coordinates selected from the group consisting of the coordinates of Tables IB, and interatomic distances and angles of active site residues listed in IIB and/or IIIB.

In yet another aspect, the invention provides a composition comprising a prenyltransferase in complex with IPP as characterized by the coordinates selected from the group consisting of the coordinates of Tables IC, and interatomic distances and angles of active site residues listed in IIC and/or IIIC.

In another aspect, the invention provides a heavy atom derivative of a Streptococcus pneumoniae UPPS crystal wherein the prenyltransferase comprises a protein having the coordinates represented in any of FIGS. 2, 3, 4 and/or 5, and listed in Tables IA-IC, IIA-IIC, and/or IIIA-IIIC.

In another aspect, the invention provides a characterized by an α+β fold with three layers, αβα, wherein the β-strands form a six-strand parallel β-sheet and three α-helices pack against one face of the sheet and three to four α-helices located on the opposite face.

In yet another aspect, the invention provides a composition comprising a Streptococcus pneumoniae UPPS in orthorhombic crystalline form having a space group of P2₁2₁2₁ wherin the lattice constants are a=59.6 Å, b=118.0 Å, c=178.2 Å and containing two 60 kDa dimers in an asymmetric unit.

In another aspect, the invention provides a composition comprising a Streptococcus pneumoniae UPPS in orthorhombic crystalline form having a space group of I2₁2₁2₁.

In yet another aspect, the invention provides a composition comprising a co-crystal of Streptococcus pneumoniae UPPS in complex with IPP in orthorhombic crystalline form having a space group selected from the group consisting of P2₁2₁2₁ and I2₁2₁2₁.

In yet another aspect, the invention provides a composition comprising a co-crystal of Streptococcus pneumoniae UPPS in complex with a substrate FPP in monoclinic crystalline form having a space group of P2₁ and the crystalline form has lattice constants of a=58.1 Å, b=44.6 Å, c=115.5 Å, β=98.7°.

In another aspect, the invention provides a process for determining a crystal structure form using structural coordinates of a Streptococcus pneumoniae UPPS crystal or portions thereof, to determine a crystal form of a mutant, homologue, or co-complex of said active site by molecular replacement.

In yet another aspect, the invention provides a process of identifying an inhibitor compound capable of binding to and inhibiting an enzymatic activity of a Streptococcus pneumoniae UPPS said process comprising: introducing into a suitable computer program information defining an active site conformation of a UPPS molecule comprising a conformation defined by coordinates listed in Table IA, IIA, and/or IIIA wherein said program displays the three-dimensional structure thereof; creating a three dimensional structure of a test compound in said computer program; displaying and superimposing a model of said test compound on a model of said active site; incorporating said test compound in a biological prenyltransferase activity assay for a prenyltransferase characterized by said active site; and determining whether said test compound inhibits enzymatic activity in said assay.

In another aspect, the invention provides a process designing drugs useful for inhibiting UPPS activity using certain or all of the atomic coordinates of a Streptococcus pneumoniae UPPS crystal to computationally evaluate a chemical entity for associating with an active site of a UPPS enzyme.

In yet another aspect, the invention provides a method of modifying a test UPPS polypeptide comprising: providing a test UPPS polypeptide sequence having a characteristic that is targeted for modification; aligning the test UPPS polypeptide sequence with at least one reference UPPS polypeptide sequence for which an X-ray structure or other structure is available, wherein the at least one reference UPPS polypeptide sequence has a characteristic that is desired for the test UPPS polypeptide; building a three-dimensional model for the test UPPS polypeptide using the three-dimensional coordinates of the X-ray structure(s) or other structure(s) of the at least one reference UPPS polypeptide and its sequence alignment with the test UPPS polypeptide sequence; examining the three-dimensional model of the test UPPS polypeptide for a difference in an amino acid residue as compared to the at least one reference polypeptide, wherein the residues are associated with the desired characteristic; and mutating an amino acid residue in the test UPPS polypeptide sequence located at a difference identified to a residue associated with the desired characteristic, whereby the test UPPS polypeptide is modified.

In another aspect, the invention provides a process of identifying an inhibitor compound capable of inhibiting an enzymatic activity of a Streptococcus pneumoniae UPPS, said process comprising: carrying out an in vitro assay by introducing said compound in a biological prenyltransferase activity assay containing a prenyltransferase of the invention; and determining whether said test compound inhibits an enzymatic activity of the prenyltransferase in said assay.

In yet another aspect, the invention provides a product of the process of identifying an inhibitor compound capable of inhibiting an enzymatic activity of a Streptococcus pneumoniae UPPS which is a peptide, peptidomimetic, or synthetic molecule and is useful for inhibiting the metallo-beta lactamase, preferably in the treatment of bacterial infections in a mammal.

In another aspect, the invention provides a product of the process of identifying an inhibitor compound capable of inhibiting an enzymatic activity of a Streptococcus pneumoniae UPPS wherein said product is a competitive or non-competitive inhibitor of the Streptococcus pneumoniae prenyltransferase.

In another aspect, the invention provides a process of designing drugs useful for inhibiting Streptococcus pneumoniae UPPS comprising using atomic coordinates of a Streptococcus pneumoniae UPPS crystal or atomic coordinates of a Streptococcus pneumoniae UPPS in complex with FPP or IPP to computationally evaluate a chemical entity for associating with an active site of a Streptococcus pneumoniae UPPS.

In another aspect, the invention provides a process of designing drugs useful for inhibiting Streptococcus pneumoniae UPPS comprising the step of using structure coordinates of Streptococcus pneumoniae UPPS to identify an intermediate in a chemical reaction between said prenyltransferase and a compound that is a substrate or inhibitor of said prenyltransferase. In another aspect, the invention provides a process of designing drugs useful for inhibiting Streptococcus pneumoniae UPPS wherein structure coordinates comprise the coordinates corresponding to any of the structures shown in FIGS. 2, 3, 4 and/or 5 and/or listed in Tables IA-IC, IIA-IIC, and/or IIIA-IIIC.

In one aspect, the present invention relates to an UPPS that is derived from Streptococcus pneumoniae and comprising a protein having the amino acid sequence shown in SEQ ID No. 1, and coordinates of Table I, and interatomic distances and angles of active site residues listed in Table II and/or III, in an essentially pure native form or a homolog thereof.

In another aspect, the present invention provides a novel crystalline form of a UPPS enzyme active site in complex with IPP, having the coordinates of Table I, and interatomic distances and angles of active site residues listed in Table II, and/or III.

In yet another aspect, the present invention provides a novel crystalline form of the UPPS enzyme active site in complex with the substrate farnesyl pyrophosphate, identified herein FPP, having the coordinates of Table I, and interatomic distances and angles of active site residues listed in Table II, and/or III.

In yet another aspect, the invention provides a model of specific roles of residues in the active site responsible for the binding of substrates, substrate analogs, and inhibitors.

In yet another aspect, the invention provides astructural basis for the role of active site amino acid residues and metals bound in an active site in a catalytic activity of these enzymes. This aspect of the invention provides a method for identifying inhibitors of a UPPS, which methods comprise the steps of: providing coordinates of a UPP structure of the invention to a computerized modeling system; identifying compounds that bind to an active site; and screening the compounds identified for undecaprenyl pyrophosphate synthase inhibitory bio-activity.

Another aspect of this invention includes machine-readable media encoded with data representing coordinates of a three-dimensional structure of a UPPS crystal structure alone or in complex with IPP and/or FPP.

Other aspects and advantages of the present invention are described further in the following detailed description of the preferred embodiments thereof.

### DESCRIPTION OF THE FIGURES

FIG. 1A provides a representation of the chemical structure of FPP, and the numbering scheme used.

FIG. 1B provides a representation of the chemical structure of IPP, and the numbering scheme used.

FIG. 2A provides a representation of the secondary structure elements of native UPPS, from Streptococcus pneumoniae.

FIG. 2B provides a representation of the topology of UPPS. Triangles denote β-strands and circles denote α-helices.

FIG. 3A provides a schematic drawing of a UPPS enzyme from Streptococcus pneumoniae in complex with IPP bound in the active site.

FIG. 3B provides a schematic drawing of a UPPS enzyme from Streptococcus pneumoniae in complex with FPP bound in the active site.

FIG. 4A provides a schematic drawing of FPP bound in an active site of UPPS from S. pneumoniae. Shown and labeled in this view are the hydrophobic side chains of residues lining an FPP binding pocket, the interactions of the phosphate groups bound at the N-terminus of α1, a tightly bound water molecule, and the role of conserved arginine residues that are important for FPP binding.

FIG. 4B provide a schematic drawing of FPP bound in the active site of UPPS from S. pneumoniae. In this view are shown, along the axis of the extended FPP molecule, the relative orientation of the important residues for catalysis, and those side chains important for substrate binding.

FIG. 5 provides a schematic drawing of the interactions between the active site residues of a UPPS from Streptococcus pneumoniae and the substrate IPP. Important for the substrate binding are arginine side chains from the C-terminus of the other molecule in a dimer. Also shown is a pyrophosphate molecule that occupies the same position as the pyrophosphate moiety of FPP.

### DETAILED DESCRIPTION OF THE INVENTION

The present invention provides a Streptococcus pneumoniae UPPS crystalline structure of a native enzyme. In addition, it provides an undecaprenyl pyrophosphate synthase active site of the crystalline structure of the UPPS, in complex with IPP and in complex with FPP and methods to use these crystalline forms and their active sites to identify and improve UPPS inhibitor compounds (peptide, peptidomimetic or synthetic compositions). These compounds are characterized by an ability to competitively inhibit binding of substrates or other like-molecules to the active site of undecaprenyl pyrophosphate synthases.

A UPPS from Streptococcus pneumoniae Crystalline Three-Dimensional Structure and its Complex with IPP and FPP

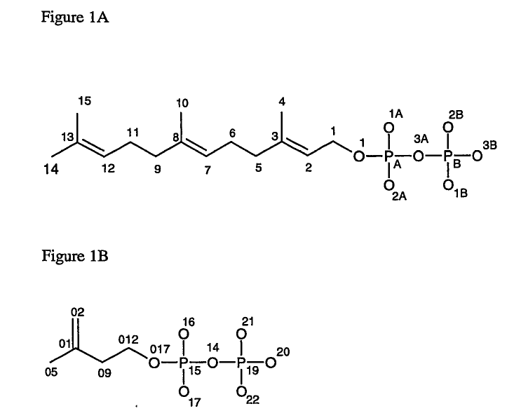

The crystal structures of a UPPS from Streptococcus pneumoniae in its native form, in complex with the substrate IPP (FIG. 1B), and a substrate, FPP (FIG. 1A) have been determined and refined to 2.3 Å 2.8 Å and 3.3 Å resolution, respectively.

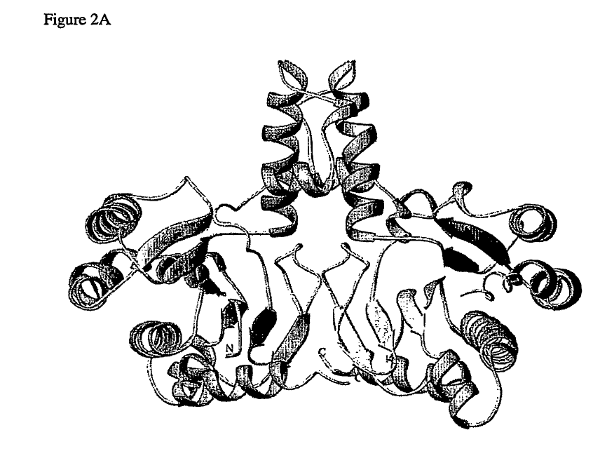

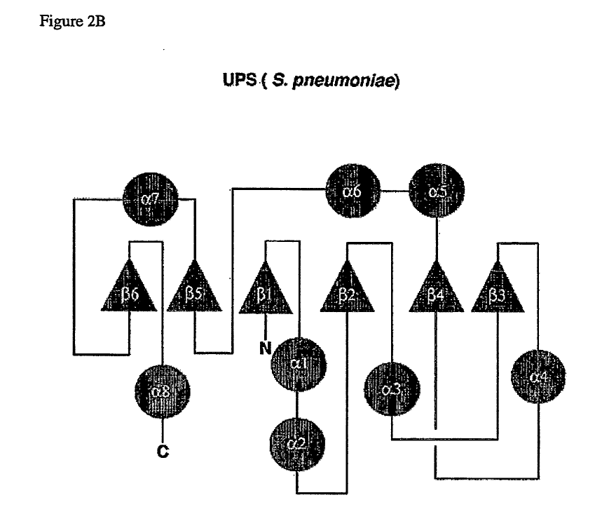

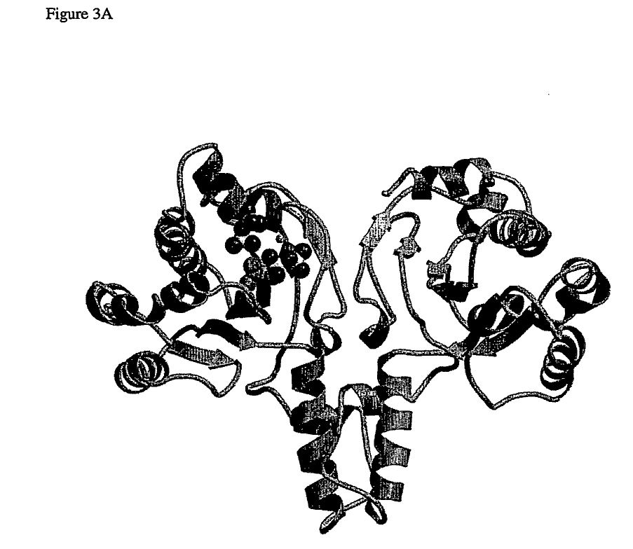

Native UPPS from Streptococcus pneumoniae crystalline structure was determined. This crystal structure consists of two homodimers in the asymmetric unit, molecules A and B form one active dimer, and molecules C and D form the second dimer. A preferred model includes residues 17-72 and 77-248 in molecule A; residues 17-72 and 79-246 in molecule B; residues 17-73 and 77-248 in molecule C; residues 18-73 and 78-246 in molecule D; and a total of 410 water molecules (FIG. 2A). Each polypeptide chain has an •+• fold characterized by three layers: •••. Four •-helices packed against one face of the central sheet, formed by six parallel •-strands with ordering 342156. Another three •-helices located at the other side of the sheet form most of the dimer interface (FIG. 2B). The central sheet is not continuous from one molecule to the other in the dimer, rather, the •-sheets from each monomer form an angle of approximately 90°. In the dimer, the N- and the C-terminus of molecule B are located near a shallow groove formed in molecule A, the proposed location of the active site suggesting that the termini on one molecule contribute to the binding of substrates in the other molecule. This may explain the need for the enzyme to be a dimer if catalysis is to occur, and the extensive 1,650 Å² of buried surface area at the dimer interface involving α5, α6, and α7. The dimer interface include interactions between three pairs of charged side chains: Arg170NH2:Asp214OD2 (3.1 Å), Arg226NE:Glu239OE2 (2.8 Å), and Glu239OE2:Arg226NE (2.6 Å). Also across the interface interact Tyr237OH and Asn229ND2 (3.1 Å).

There are three buried charged residues: Glu 152, Arg200, His22. E152 is at the N-terminus of α5 and contacts Wat146 (2.9 Å), Tyr147OH (2.6 Å), Gln214OE1 (3.2 Å), Phe184N (2.8 Å), and Gln214OE1 (3.1 Å). Arg200 is located at the C-terminus of β5 contacting with Ser218 OG (2.7 Å), Arg216O (2.9 Å), Asp46OD2 (3.1 Å), and Wat410 (2.8 Å). His22 is makes H-bonds with Leu192O (2.6 Å) and Thr68OG1 (2.9 Å).

There are five relatively small, and empty internal cavities. One cavity is next to Arg200, a residue presumably located in the active site. This cavity is bound by atoms in residues Ile198, Ile199, Arg200, Leu20, Leu22, Tyr221 and Phe222. Two more cavities are located on either side of a-helix α3, one is lined by Leu90, Pro91, Phe94, Tyr95, Val99, Ile109, Ala125, Leu126, Ala129, and Phe143. The cavity on the other side of α3 is lined by His45, Phe86, Met49, Leu90, Leu52, Asn30, Ala71, Met27, and Trp227. The presence of these two cavities suggests that the helix may be displaced upon substrate binding and for product release or membrane association. Another cavity is located C-terminal to α6 and a portion of β3 (His187, Phe184, Glu110, Ile112, Asn142, Met111) along the groove on the side of α5. This groove probably accommodates the product. N-terminal to β5 is the last cavity (Asp191; Leu192, Arg193, Asp194, Pro195, Asp196) that may allow the α6-β5 connection slide sideways or back and forth when the product is being processed during catalysis. No solvent molecules have been found occupying these cavities.

In all four molecules in the asymmetric unit, residues 74-76 are disordered in a S. pneumoniae native UPPS structure, and residues in the immediate vicinity have higher B-factors than the average of the structure suggesting that these residues are highly mobile in the absence of ligands or substrates, and that they may be part of the active site. In addition, based on the alignment of UPPS amino acid sequences, the most conserved residues are located in the short α-helix α1 (residues 28-32) and α2-β3 (residues 73-83), and in the β5-α7 (residues 200-214) loops suggesting that the active site is located at the top of the sheet in a shallow groove, next to the disordered region. As discussed below, the crystal structures of the UPPS complexes with the substrates FPP and IPP confirm the location of the active site, the critical role played by the conserved residues for substrate binding and their role in catalysis, and show that the disordered residues become ordered upon substrate binding.

The undecaprenyl pyrophosphate synthases from S. pneumoniae and M. luteus share a 37% amino acid sequence identity and the polypeptide have the same fold. Superposition of the Cα atoms of the M. luteus and S. pneumoniae native UPPS crystal structures results in an overall root mean square, rms, deviation of 0.6 Å.

The present invention also provides a novel undecaprenyl pyrophosphate synthase crystalline structure based on the UPPS Streptococcus pneumoniae undecaprenyl pyrophosphate synthase in complex with the substrates IPP and FPP.

In the FPP complex, one FPP molecule is bound in the active site. However, in the IPP complex structure, two large, adjacent electron density peaks appear in the active site, the first one corresponds to a pyrophosphate onto which the pyrophosphate of FPP can be superimposed. The second electron density peak corresponds to an entire IPP molecule. In both complex structures, the major structural changes between the native and the substrate complex structures is the ordering of the polypeptide chain between residues 72-79, to form two turns of a 3₁₀ helix and the opening of the entrance to the long, narrow hydrophobic pocket where FPP binds. In the IPP complex, the C-terminus of the other molecule in the dimer also becomes ordered to form part of the IPP binding site.

The interactions between the substrates and the enzyme are summarize as follows:

1. There are at least two substrate-binding sites, at least one of each corresponding to each of the two substrates. The FPP's pyrophosphate binds at the N-terminus of •-helix •1 and the C-terminal end of strands •1 and •2. The farnesyl carbon chain runs across •2 and •3. The IPP's pyrophosphate binding site is located next to an FPP site and also runs across the top strands •1 and •2, but on the other side of the •-sheet. The phosphate group interacts with Arg247 located on the C-terminus of the partner molecule in the dimer

2. The farnesyl chain binds into a tunnel lined by Met49, Leu52, Ala71, Leu90, Pro91, Phe94, Leu126, Phe143, and Leu145.

3. The isopentenyl chain binds into a shallow depression lined by Ile26, the C• carbon of Asp28, Tyr70, and Phe72. The isopentenyl chain is then substantially or completely enclosed by the farnesyl chain bound adjacent to it and by the C-terminus of the partner molecule in the dimer. They are bound in such a way that the re face of an attacking carbon is poised for the reaction.

4. The position of the Mg⁺² ions in a metal binding site may be indicated by a strong difference in an electron density peak modeled as a water molecule is located between the two pyrophosphate groups observed bound in the IPP complex.

5. The enzymatic turnover cycle starts with binding of FPP required for a subsequent binding of IPP. IPP binding must follow FPP because a binding interaction is with magnesium that is bridging the two pyrophosphates. Also, the carbon chain of FPP forms part of the IPP binding pocket. The orientation of C02 of IPP is such, that C1 of FPP is facing the re face of the double bond, ideal for the attack on C1. This is necessary, since the removal of a proton by Asp28 (or Arg200) occurs from the opposite side (on C9) to produce a Z-double bond.

6. A preferred mechanism provides a critical role for His45, in •2, to promote cleavage of the pyrophosphate moiety from FPP by positioning the NE2 atom to polarize C1 in FPP, in analogy to a mechanism of thiamine phosphate synthase. Another preferred mechanism involves a metal-triggered carbocation formation. Once an FPP is bound, the binding of IPP and Mg+2 result in the formation of a carbocation analogously to a mechanism postulated for other prenyltransferases (i.e., farnesyl synthase, or aristolochene synthase). In addition to His45, Asn30 and/or Asp28, or Arg200 in •1 is putatively important for a stereochemically-specific removal of a proton to form a double bond. These residues occupy suitable relative positions for catalysis as deduced from a comparison of complex structures. Asn30, Asp28 or Arg200 are poised to assist a stereochemical-specific proton abstraction from an incoming isopentenyl unit from C09. These residues are highly conserved among UPPSs from a variety of organisms. Of 27 aligned amino acid sequences including bacterial, archaebacteria and eukariotic UPPS's, the stretch FGHKA that includes Gly44 is absolutely conserved while His45 is present in all but two known sequences. In these two known sequences, a tyrosine replaces the histidine. In the same alignment, an amino acid stretch that includes Asp28, IMDGN, Asp28, Gly29 and Asn30 is absolutely conserved. Site-directed mutagenesis of some conserved residues of UPPS from E. coli (Pan, J-J., Yang, L.-W., and Liang, P-H. (2000) Biochemistry 39, 13856-13861) can now be rationally explained by the discoveries of the present invention. Mutation of Asp26 or Glu213 to alanine causes a 1000-fold drop in k_{cat}. In S. pneumoniae Asp28 is equivalent to E. coli 's Asp26. In a preferred catalytic mechanism provided by the invention, Asp28/Asp26 plays a key role in formation of a double bond in the product after condensation step has occurred by removing a proton that would result in a cis (Z) configuration. A second important mutation involves Glu213, in S. pneumoniae Glu219 is equivalent to E. coli 's Glu213. Glu219/Glu213 interacts with and helps to position Arg206 in aproper orientation to interact with IPP in an active site. Arg206 is an important residue for binding of IPP.

Certain mechanisms of the invention take into account the binding of the product's chain when the product exceeds 20 carbons. A model of a C30 intermediate having the preferred stereochemistry (trans, trans, cis, cis, cis) bound at the FPP binding site in an active site UPPS shows that the chain beyond the first 15 carbon atoms can exit the protein into the solvent or, more likely, into a phospholipid membrane or detergent micell through a opening created between helices •2 and •3. Creation of an opening requires the movement of side chains of Met49 and Tyr98, torsion of the side chains is sufficient to open a channel through which a product may exit. There is also the possibility, as has been suggested, that a product may fold on itself several times inside an FPP binding channel, but there does not seem possible since there is not enough space to accommodate a long carbon chain. The final product exits the protein by either a “pull” action from the pyrophosphate end, or by a “pull” action from the opposite end, but that implies that the pyrophosphate is dragged along an FPP binding channel and out through an inter-helix space.

Table I provides the atomic coordinates of preferred native and complex crystal structures of UPPS from S. pneumoniae. This preferred native model includes residues 37-92 and 97-268 in molecule A, 37-92 and 99-266 in molecule B, 37-93 and 97-268 in molecule C, 38-93 and 98-266 in molecule D, for the four molecules, A, B, C, and D, in the crystallographic asymmetric unit. The amino acid sequence of a UPPS from S. pneumoniae is provided in SEQ ID No. 1. Table II provides the distances, in Å, between atoms within a 5.0 Å radius in anactive site including bound substrates FPP and IPP. Table III provides the angles (°) between active site atoms at are within 4.0 Å of substrate FPP or IPP.

Small variations in the atomic coordinates shown in Tables I-III will occur such as upon refinement of a crystal structure from a different crystal form that will result in a new set of coordinates. The deviation on Cα atoms from the present coordinate set is not expected to substantially exceed a rms of 2.5 Å. Similarly, bond angles and bond lengths will usually vary within a small range (Engh, R. A., and Huber, R. (1991) Acta Crystallogr. A47, 392-400), however, the inter-atomic interactions in Tables I-III will remain constant, within the experimental error, as will the relative conformation and orientation or positioning of residues in an active site. The atomic coordinates of an active site residues, including bound substrates, are provided in Tables I-III.

Mutants and Derivatives

Herein, the terms “a” and “an” mean “one or more” when used in this application, including the claims.

The invention further provides homologues, co-complexes, mutants and derivatives of the UPPS crystal structure of the invention.

The term “homologue” means a protein having at least 30% amino acid sequence identity with a functional domain of UPPS. Preferably the percentage identity will be 40, or 50%, more preferably 60 or 70% and most preferably 80 or 90%. A 95% identity is most particularly preferred.

The term “co-complex” means a UPPS or a mutant or homologue of a UPPS in covalent or non-covalent association with a chemical entity or compound.

As used herein, the term “agonist” means an agent that supplements or potentiates the bioactivity of a functional UPPS gene or protein or of a polypeptide encoded by a gene that is up- or down-regulated by a UPPS polypeptide. By way of specific example, an “agonist’ is a compound that interacts with a steroid hormone receptor to promote a transcriptional response. An agonist can induce changes in a receptor that places a receptor in an active conformation that allows them to influence transcription, either positively or negatively. There can be several different ligand-induced changes in a receptor's conformation. The term “agonist” specifically encompasses partial agonists.

As used herein, the terms “•-helix”, “alpha-helix” and “alpha helix” are used interchangeably and mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 mm along the long axis.

As used herein, the term “antagonist” means an agent that decreases or inhibits a bioactivity of a functional UPPS gene or protein, or that supplements or potentiates a bioactivity of a naturally occurring or engineered non-functional UPPS gene or protein. Alternatively, an antagonist can decrease or inhibit a bioactivity of a functional gene or polypeptide encoded by a gene that is up- or down-regulated by a UPPS polypeptide. An antagonist can also supplement or potentiate the bioactivity of a naturally occurring or engineered non-functional gene or polypeptide encoded by a gene that is up- or down-regulated by a UPPS polypeptide. By way of specific example, an “antagonist” is a compound that interacts with a steroid hormone receptor to inhibit a transcriptional response. An antagonist can bind to a receptor but fail to induce conformational changes that alter a receptor's transcriptional regulatory properties or physiologically relevant conformations. Binding of an antagonist can also block the binding and therefore the actions of an agonist. The term “antagonist” specifically encompasses partial antagonists.

As used herein, the terms “•-sheet”, “beta-sheet” and “beta sheet” are used interchangeably and mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the term “binding pocket” refers to any moiety, part or region of UPPS that actually or is capable of binding to, directly participating with, adhering to, or otherwise associating with an atom, ion or molecule. Preferably, a large cavity within a UPPS ligand binding domain where an agonist or antagonist can bind is a binding pocket. Such a cavity can be empty, or can contain water molecules or other molecules from the solvent, or an agonist or antagonist moieties, atoms or molecules. Such a binding pocket also includes regions of space near the “main” binding pocket that are not occupied by atoms or moieties of UPPS, but that are near the “main” binding pocket, and that are contiguous with the “main” binding pocket. Preferably,

As used herein, the term “active site” refers to a specific region of UPPS binding pocket where a molecule binds and catalysis takes place. It is comprised and bound by amino acid residues that are in direct contact with the substrate or that interact with the substrate(s) through water molecules or those amino acids that, although not being in direct contact with the substarte(s), nonetheless are important for they allow the correct positioning of those amino acids that are and which without the correct positioning they would not be able to interact favorably (i.e. in a way conducent to catalysis) with the substrate(s). These interactions between amino acids and substrate(s) are responsible for the binding of the substrate to UPPS, for the correct positioning of the substrate for catalysis, and for stabilization of any reaction intermediates and for the binding and possibly the release of the products from that active site. These are amino acids that may be replaced by site-directed mutagenesis, and their replacement will result in at the very least a several-fold, or more likely, in several orders of magnitude decrease in the binding affinity of the substrate(s). The active site is also comprised by amino acids that are directly responsible for catalysis. These amino acids interact with the substrate(s) through hydrogen bonds or are in close proximity to electron-donor or electron-acceptor centers in the substrate. These amino acids may act themselves as electron-donor or electron-acceptor centers for catalysis to take place. These are amino acids that may be replaced by site-directed mutagenesis, and their replacement will result in at the very least a several-fold, or more likely, in several orders of magnitude decrease in the catalytic efficiency, but no changes in the affinity of binding of the substarte(s). In some cases, the catalytic activity may be recovered by some chemicals that, by binding to the appropriate active site residues, will mimic the wild-type amino acid.

As used herein, the term “biological activity” means any observable effect flowing from interaction between a UPPS polypeptide and an agonist or antagonist. Representative, but non-limiting, examples of biological activity in the context of the present invention include transcription regulation, agonist or antagonist binding, and peptide binding.

As used herein, the terms “candidate substance” and “candidate compound” are used interchangeably and refer to a substance that is believed to interact with another moiety, for example an agonist or antagonist that is believed to interact with a complete, or a fragment of, a UPPS polypeptide, and which can be subsequently evaluated for such an interaction. Representative candidate substances or compounds include xenobiotics such as drugs and other therapeutic agents, carcinogens and environmental pollutants, natural products and extracts, as well as endobiotics such as glucocorticosteroids, steroids, fatty acids and prostaglandins. Other examples of candidate compounds that can be investigated using the methods of the present invention include, but are not restricted to, agonists and antagonists of a UPPS polypeptide, toxins and venoms, viral epitopes, hormones (e.g., glucocorticosteroids, opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the terms “cells,” “host cells” or “recombinant host cells” are used interchangeably and mean not only to a particular subject cell, but also to any progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “chimeric protein” or “fusion protein” are used interchangeably and mean a fusion of a first amino acid sequence encoding a UPPS polypeptide with a second amino acid sequence defining a polypeptide domain foreign to, and not homologous with, any domain of a UPPS polypeptide. A chimeric protein can include a foreign domain that is found in an organism that also expresses the first protein, or it can be an “interspecies” or “intergenic” fusion of protein structures expressed by different kinds of organisms. In general, a fusion protein can be represented by the general formula X-UPPS-Y, wherein UPPS represents a portion of the protein which is derived from a UPPS polypeptide, and X and Y are independently absent or represent amino acid sequences which are not related to a UPPS sequence in an organism, which includes naturally occurring mutants.

As used herein, the term “detecting” means confirming the presence of a target entity by observing the occurrence of a detectable signal, such as a radiologic or spectroscopic signal that will appear exclusively in the presence of the target entity.

As used herein, the term “DNA segment” means a DNA molecule that has been isolated free of total genomic DNA of a particular species. In a preferred embodiment, a DNA segment encoding a UPPS polypeptide refers to a DNA segment that comprises any of SEQ ID NO:1, but can optionally comprise fewer or additional nucleic acids, yet is isolated away from, or purified free from, total genomic DNA of a source species, such as Streptococcus pnuemoniae. Included within the term “DNA segment” are DNA segments and smaller fragments of such segments, and also recombinant vectors, including, for example, plasmids, cosmids, phages, viruses, and the like.

As used herein, the term “DNA sequence encoding a UPPS polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “expression” generally refers to the cellular processes by which a biologically active polypeptide is produced.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “crystal lattice” means the array of points defined by the vertices of packed unit cells.

As used herein, “hexagonal unit cell” means a unit cell wherein a=b•c; and •=•=90, •=120°. The vectors a, b, and c describe the unit cell edges and the angles •, •, and • describe the unit cell angles. In a preferred embodiment of the present invention, the unit cell has lattice constants of a=59.6 Å, b=118.0 Å, c=178.2 Å. While preferred lattice constants are provided, a crystalline polypeptide of the present invention also comprises variations from the preferred lattice constants, wherein the varations range from about one to about two percent.

As used herein, the term “hybridization” means the binding of a probe molecule, a molecule to which a detectable moiety has been bound, to a target sample.

As used herein, the term “interact” means detectable interactions between molecules, such as can be detected using, for example, a yeast two hybrid assay. The term “interact” is also meant to include “binding” interactions between molecules. Interactions can, for example, be protein-protein or protein-nucleic acid in nature.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “labeled” means the attachment of a moiety, capable of detection by spectroscopic, radiologic or other methods, to a probe molecule.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “modulate” means an increase, decrease, or other alteration of any or all chemical and biological activities or properties of a wild-type or mutant UPPS polypeptide, preferably a wild-type or mutant UPPS polypeptide. The term “modulation” as used herein refers to both up-regulation (i.e., activation or stimulation) and down-regulation (i.e. inhibition or suppression) of a response, and includes responses that are upregulated in one cell type or tissue, and down-regulated in another cell type or tissue.

As used herein, the term “molecular replacement” means a method that involves generating a preliminary model of a wild-type UPPS ligand binding domain, or a UPPS mutant crystal whose structure coordinates are unknown, by orienting and positioning a molecule or model whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol., 115: 55-77; Rossmann, ed, (1972) The Molecular Replacement Method, Gordon & Breach, New York. Using the structure coordinates of the active site of UPPS provided by this invention, molecular replacement can be used to determine the structure coordinates of a crystalline mutant or homologue of the UPPS active site, or of a different crystal form of the UPPS active site.

As used herein, the term “partial agonist” means an entity that can bind to a receptor and induce only part of the changes in the receptors that are induced by agonists. The differences can be qualitative or quantitative. Thus, a partial agonist can induce some of the conformation changes induced by agonists, but not others, or it can only induce certain changes to a limited extent.

As used herein, the term “partial antagonist” means an entity that can bind to a receptor and inhibit only part of the changes in the receptors that are induced by antagonists. The differences can be qualitative or quantitative. Thus, a partial antagonist can inhibit some of the conformation changes induced by an antagonist, but not others, or it can inhibit certain changes to a limited extent.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “sequencing” means the determining the ordered linear sequence of nucleic acids or amino acids of a DNA or protein target sample, using conventional manual or automated laboratory techniques.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of coordinates determined by X-ray crystallography is not without standard error. In general, the error in the coordinates tends to be reduced as the resolution is increased, since more experimental diffraction data is available for the model fitting and refinement. Thus, for example, more diffraction data can be collected from a crystal that diffracts to a resolution of 2.8 angstroms than from a crystal that diffracts to a lower resolution, such as 3.5 angstroms. Consequently, the refined structural coordinates will usually be more accurate when fitted and refined using data from a crystal that diffracts to higher resolution. The design of agonists, antagonists, and modulators for UPPS depends on the accuracy of the structural coordinates. If the coordinates are not sufficiently accurate, then the design process will be ineffective. In most cases, it is very difficult or impossible to collect sufficient diffraction data to define atomic coordinates precisely when the crystals diffract to a resolution of only 3.5 angstroms or poorer. Thus, in most cases, it is difficult to use X-ray structures in structure-based agonist and antagonist design when the X-ray structures are based on crystals that diffract to a resolution of only 3.5 angstroms or poorer. However, common experience has shown that crystals diffracting to 2.8 angstroms or better can yield X-ray structures with sufficient accuracy to greatly facilitate structure-based drug design. Further improvement in the resolution can further facilitate structure-based design, but the coordinates obtained at 2.8 angstroms resolution are generally adequate for most purposes.

Also, those of skill in the art will understand that UPPS proteins can adopt different conformations when different agonists, antagonists, and modulators are bound. Subtle variations in the conformation can also occur when different agonists are bound, and when different antagonists are bound. These variations can be difficult or impossible to predict from a single X-ray structure. Generally, structure-based design of UPPS modulators depends to some degree on a knowledge of the differences in conformation that occur when agonists and antagonists are bound. Thus, structure-based modulator design is most facilitated by the availability of X-ray structures of complexes with potent agonists as well as potent antagonists.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which is it associated in nature.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “transcription factor” means a cytoplasmic or nuclear protein which binds to such gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the term “unit cell” means a basic parallelipiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles •, •, and • define the angles between the vectors: angle • is the angle between vectors b and c; angle • is the angle between vectors a and c; and angle • is the angle between vectors a and b. The entire volume of a crystal can be constructed by regular assembly of unit cells; each unit cell comprises a complete representation of the unit of pattern, the repetition of which builds up the crystal.

As used herein, the term “mutant” or “mutation” carries its traditional connotation and means a change, inherited, naturally occurring or introduced, in a nucleic acid or polypeptide sequence, and is used in its sense as generally known to those of skill in the art. For example, a UPPS polypeptide, i.e., a polypeptide displaying the biological activity of wild-type UPPS activity, characterized by the replacement of at least one active-site amino acid from the wild-type prenyltransferase sequence. Such a mutant may be prepared, for example, by expression of the UPPS prenyltransferase cDNA previously altered in its coding sequence by oligonucleotide-directed mutagenesis.

UPPS mutants may also be generated by site-specific incorporation of unnatural amino acids into the UPPS protein using the general biosynthetic method of C. J. Noren et al, Science, 244:182-188 (1989). In this method, the codon encoding the amino acid of interest in wild-type UPPS is replaced by a “blank” nonsense codon, TAG, using oligonucleotide-directed mutagenesis. A suppressor directed against this codon is then chemically aminoacylated in vitro with the desired unnatural amino acid. The aminoacylated residue is then added to an in vitro translation system to yield a mutant UPPS enzyme with the site-specific incorporated unnatural amino acid.

Selenocysteine or selenomethionine may be incorporated into wild-type or mutant metallo UPPS prenyltransferase by expression of UPPS-encoding cDNAs in auxotrophic E. coli strains (W. A. Hendrickson et al, EMBO J., 9(5):1665-1672 (1990)) or a normal strain grown in a medium supplemented with appropriate nutrients that will prevent endogenous synthesis of methionine. In either of these methods, the wild-type or mutated undecaprenyl pyrophosphate synthase cDNA may be expressed in a host organism on a growth medium depleted of either natural cysteine or methionine (or both) but enriched in selenocysteine or selenomethionine (or both).

The term “heavy atom derivative” refers to derivatives of UPPS produced by chemically modifying a crystal of UPPS. In practice, a native crystal is tretaed by immersing it in a solution containing the desired metal salt, or organometallic compound, e.g., lead chloride, gold thiomalate, thimerosal or uranyl acetate, which upon diffusion into the protein crystal can bind to the protein. The location of the bound heavy metal atom site(s) can be determined by X-ray diffraction analysis of the treated crystal. This information, in turn, is used to generate the phase angle information needed to construct a three-dimensional electron density map from which a model of the atomic structure of the enzyme is derived (T. L. Blundel and N. L. Johnson, Protein Crystallography, Academic Press (1976)).

The term “space group” refers to the arrangement of symmetry elements (i.e. molecules) throughout the crystal. There are only 132 possible arrangements, each one unique and identified by a symbol. The space group symbol is formed by a letter (P, F, I, C) and numbers with or without subscripts, for example: P2₁, I222, C2₁2₁2₁, etc.

Methods of Identifying Inhibitors of UPPS from Streptococcus pneumoniae Crystalline Structure

An aspect of this invention involves a method for identifying inhibitors of a UPPS characterized by the crystal structure and novel active site described herein, and the crystal structures of the complexes with its substrates. The novel prenyltransferase crystalline structure of the invention permits the identification of inhibitors of prenyltransferase activity. Such inhibitors may be competitive, binding to all or a portion of the active site of UPPS; or non-competitive and bind to and inhibit undecaprenyl pyrophosphate synthase whether or not it is bound to another chemical entity.

One design approach is to probe a UPPS crystal of the invention with molecules composed of a variety of different chemical entities to determine optimal sites for interaction between candidate UPPS inhibitors and the enzyme. For example, high resolution X-ray diffraction data collected from crystals saturated with solvent allows the determination of where each type of solvent molecule binds. Small molecules that bind tightly to those sites can then be designed and synthesized and tested for a UPPS inhibitor activity (J. Travis, Science, 262:1374 (1993)).

This invention also enables the development of compounds that can isomerize to short-lived reaction intermediates in the chemical reaction of a substrate or other compound that binds to or with a UPPS. Thus, the time-dependent analysis of structural changes in a UPPS during its interaction with other molecules is permitted. The reaction intermediates of the UPPS can also be deduced from the reaction product in co-complex with a UPPS. Such information is useful to design improved analogues of known UPPS inhibitors or to design novel classes of inhibitors based on the reaction intermediates of a UPPS enzyme and UPPS inhibitor co-complex. This provides a novel route for designing UPPS inhibitors with both high specificity and stability.

Another approach made possible by this invention, is to screen computationally small molecule data bases for chemical entities or compounds that can bind in whole, or in part, to a UPPS enzyme. In this screening, the quality of fit of such entities or compounds to the binding site may be judged either by shape complementarity or by estimated interaction energy (E. C. Meng et al, J. Comp. Chem., 13:505-524 (1992)).

Because UPPS may crystallize in more than one crystal form, the structure coordinates of UPPS, or portions thereof, as provided by this invention are particularly useful to solve the structure of those other crystal forms of UPPS. They may also be used to solve the structure of UPPS mutant co-complexes, or of the crystalline form of any other protein with significant amino acid sequence homology to any functional domain of UPPS.

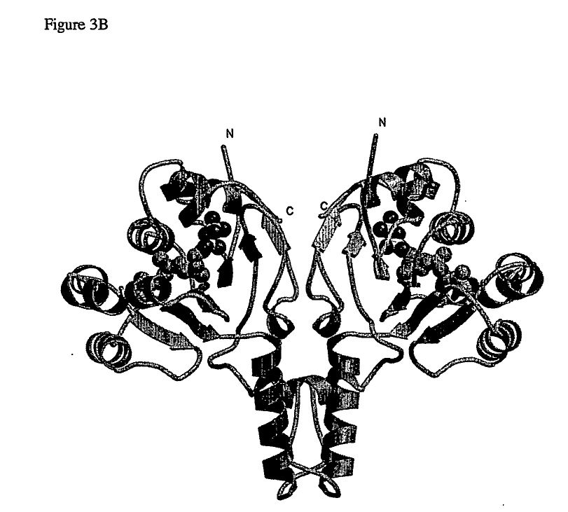

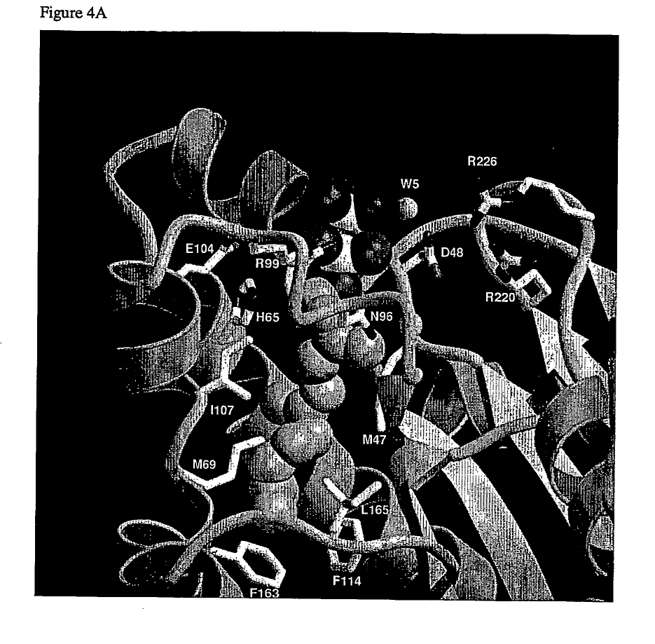

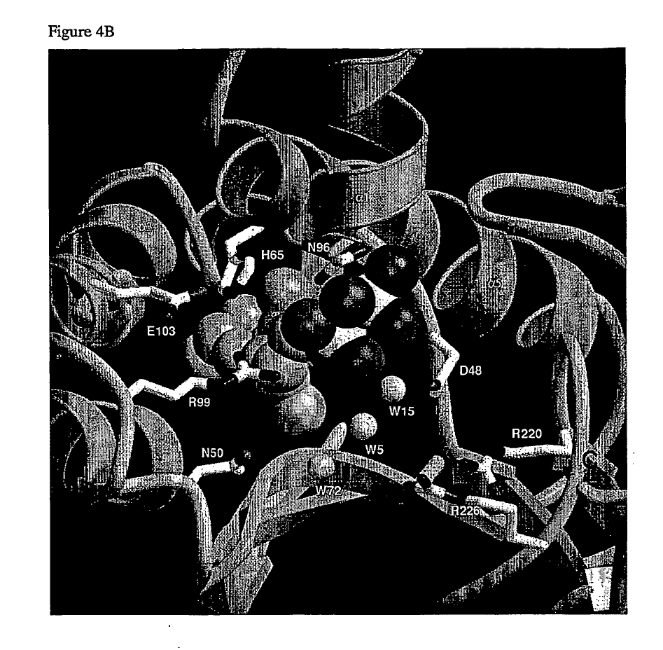

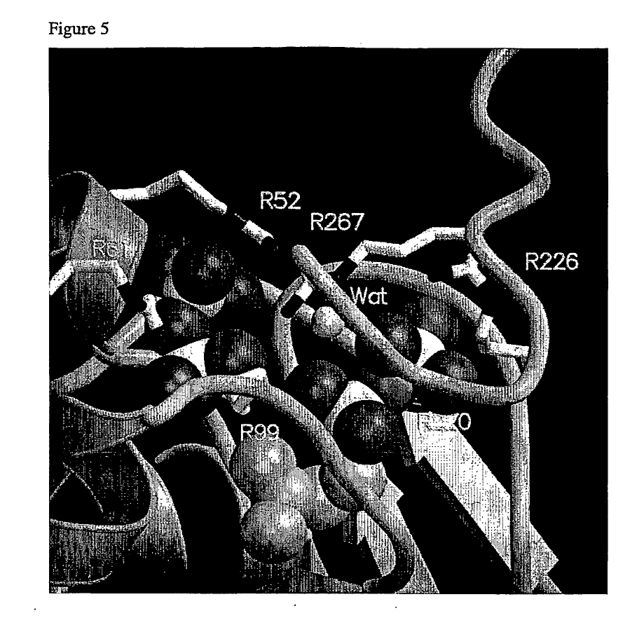

One method that may be employed for this purpose is molecular replacement. In this method, the unknown crystal structure, whether it is another crystal form of UPPS, a UPPS mutant, a UPPS co-complex, a UPPS from a different bacterial species, or the crystal of some other protein with significant amino acid sequence homology to any domain of UPPS, may be determined using the UPPS structure coordinates of this invention as provided in FIGS. 1-5 and Tables I-III. This method will provide an accurate structural form for the unknown crystal more quickly and efficiently than attempting to determine such information ab initio.

Thus, preferred UPPS structures provided herein permits the screening of known molecules and/or the designing of new molecules which bind to the structure, particularly at the binding pocket or active site, via the use of computerized evaluation systems. For example, computer modeling systems are available in which the sequence of a UPPS, and a UPPS structure (i.e., the atomic coordinates, bond distances between atoms in the active site region, etc. as provided by Tables I-III herein) may be input. Thus, a machine readable medium may be encoded with data representing the coordinates of Tables I-III. The computer then generates structural details of the site into which a test compound should bind, thereby enabling the determination of the complementary structural details of said test compound.

More particularly, the design of compounds that bind to or inhibit UPPS according to this invention generally involves consideration of two factors. First, the compound must be capable of physically and structurally associating with UPPS. Non-covalent molecular interactions important in the association of UPPS with its substrate include hydrogen bonding, van der Waals, and hydrophobic interactions.

Second, the compound must be able to assume a conformation that allows it to associate with UPPS. Although certain portions of the compound will not directly participate in this association with UPPS, those portions may still influence the overall conformation of the molecule. This, in turn, may have a significant impact on potency. Such conformational requirements include the overall three-dimensional structure and orientation of the chemical entity or compound in relation to all or a portion of the binding site, e.g., binding pocket, active site, or substrate binding sites of UPPS, or the spacing between functional groups of a compound comprising several chemical entities that directly interact with UPPS.

Another approach made possible by this invention is to screen computationally small molecule databases for chemical entities or compounds that can bind in whole, or in part, to a UPPS enzyme. Details on this process and the results it can provide are now documented in the art. For a description of this type of technology please refer to PCT application WO 97/16177 published 9 May 1997; the techniques described there for computer modeling are incorporated herein by reference.

Once identified by the modeling techniques, the prenyltransferase inhibitor may be tested for bio-activity using standard techniques. For example, the structure of the invention may be used in enzymatic activity assays to determine the inhibitory activity of the compounds or binding assays using conventional formats to screen inhibitors. One particularly suitable assay format includes the enzyme-linked immunosorbent assay (herein “ELISA”). Other assay formats may be used; these assay formats are not a limitation on the present invention.

The potential inhibitory or binding effect of a chemical compound on UPPS may be analyzed prior to its actual synthesis and testing by the use of computer modelling techniques. If the theoretical structure of the given compound suggests insufficient interaction and association between it and UPPS, synthesis and testing of the compound is obviated. However, if computer modelling indicates a strong interaction, the molecule may then be synthesized and tested for its ability to bind to UPPS and inhibit using a suitable assay. In this manner, synthesis of inoperative compounds may be avoided.

An inhibitory or other binding compound of UPPS may be computationally evaluated and designed by means of a series of steps in that chemical entities or fragments are screened and selected for their ability to associate with the individual binding pockets or other areas of UPPS.

One skilled in the art may use one of several methods to screen chemical entities or fragments for their ability to associate with UPPS and more particularly with the individual binding pockets of the UPPS active site or accessory binding site. This process may begin by visual inspection of, for example, the active site on the computer screen based on the UPPS coordinates in Tables I-III. Selected fragments or chemical entities may then be positioned in a variety of orientations, or docked, within an binding pocket or active site of UPPS. Docking may be accomplished using software such as Quanta and Sybyl, followed by energy minimization and molecular dynamics with standard molecular mechanics forcefields, such as CHARMM and AMBER.

Specialized computer programs may also assist in the process of selecting fragments or chemical entities. These include:
1. GRID (P. J. Goodford, “A Computational Procedure for Determining Energetically Favorable Binding Sites on Biologically Important Macromolecules”, J. Med. Chem., 28:849-857 (1985)). GRID is available from Oxford University, Oxford, UK.
2. MCSS (A. Miranker and M. Karplus, “Functionality Maps of Binding Sites: A Multiple Copy Simultaneous Search Method”, Proteins: Structure, Function and Genetics, 11:29-34 (1991)). MCSS is available from Molecular Simulations, Burlington, Mass.
3. AUTODOCK (D. S. Goodsell and A. J. Olsen, “Automated Docking of Substrates to Proteins by Simulated Annealing”, Proteins: Structure, Function, and Genetics, 8:195-202 (1990)). AUTODOCK is available from Scripps Research Institute, La Jolla, Calif.
4. DOCK (I. D. Kuntz et al., “A Geometric Approach to Macromolecule-Ligand Interactions”, J. Mol. Biol., 161:269-288 (1982)). DOCK is available from University of California, San Francisco, Calif.

In addition, other commercially available computer databases for small molecular compounds includes Cambridge Structural Database, Fine Chemical Database, and CONCORD, for a review see Rusinko, A., Chem. Des. Auto. News 8, 44-47 (1993).

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of UPPS. This would be followed by manual model building using software such as Quanta or Sybyl.

Useful programs to aid one of skill in the art in connecting the individual chemical entities or fragments include:
1. CAVEAT (P. A. Bartlett et al, “CAVEAT: A Program to Facilitate the Structure-Derived Design of Biologically Active Molecules”, in Molecular Recognition in Chemical and Biological Problems”, Speical Pub., Royal Chem. Soc. 78, pp. 182-196 (1989)]. CAVEAT is available from the University of California, Berkeley, Calif.
2. 3D Database systems such as MACCS-3D (MDL Information Systems, San Leandro, Calif.). This area is reviewed in Y. C. Martin, “3D Database Searching in Drug Design,” J. Med. Chem., 35:2145-2154 (1992).
3. HOOK (available from Molecular Simulations, Burlington, Mass.).

Instead of proceeding to build a UPPS inhibitor in a step-wise fashion one fragment or chemical entity at a time as described above, inhibitory or other UPPS binding compounds may be designed as a whole or “de novo” using either an empty active site or optionally including some portion(s) of a known ligand(s). These methods include:
1. LUDI (H. J. Bohm, “The Computer Program LUDI: A New Method for the De Novo Design of Enzyme Inhibitors”, J. Comp. Aid. Molec. Design, 6:61-78 (1992)). LUDI is available from Biosym Technologies, San Diego, Calif.
2. LEGEND (Y. Nishibata and A. Itai, Tetrahedron, 47:8985 (1991)). LEGEND is available from Molecular Simulations, Burlington, Mass.
3. LeapFrog (available from Tripos Associates, St. Louis, Mo.).

Other molecular modelling techniques may also be employed in accordance with this invention. See, e.g., N. C. Cohen et al, “Molecular Modeling Software and Methods for Medicinal Chemistry”, J. Med. Chem., 33:883-894 (1990). See also, M. A. Navia and M. A. Murcko, “The Use of Structural Information in Drug Design”, Current Opinions in Structural Biology, 2:202-210 (1992). For example, where the structures of test compounds are known, a model of the test compound may be superimposed over the model of the structure of the invention. Numerous methods and techniques are known in the art for performing this step, any of which may be used. See, e.g., P. S. Farmer, Drug Design, Ariens, E. J., ed., Vol. 10, pp 119-143 (Academic Press, New York, 1980); U.S. Pat. No. 5,331,573; U.S. Pat. No. 5,500,807; C. Verlinde, Structure, 2:577-587 (1994); and I. D. Kuntz, Science, 257:1078-1082 (1992). The model building techniques and computer evaluation systems described herein are not a limitation on the present invention.

Thus, using these computer evaluation systems, a large number of compounds may be quickly and easily examined and expensive and lengthy biochemical testing avoided. Moreover, the need for actual synthesis of many compounds is effectively eliminated.

In another aspect, the undecaprenyl pyrophosphate synthase structure of the invention permit the design and identification of synthetic compounds and/or other molecules which are characterized by the conformation of the undecaprenyl pyrophosphate synthase of the invention. Using known computer systems, the coordinates of the undecaprenyl pyrophosphate synthase structure of the invention may be provided in machine readable form, the test compounds designed and/or screened and their conformations superimposed on the structure of the undecaprenyl pyrophosphate synthase of the invention. Subsequently, suitable candidates identified as above may be screened for the desired undecaprenyl pyrophosphate synthase inhibitory bio-activity, stability, and the like.

Once identified and screened for biological activity, these inhibitors may be used therapeutically or prophylactically to block undecaprenyl pyrophosphate synthase activity, and thus, overcome bacterial resistance to antibiotics, for example, of the beta-lactam class, eg. imipenem, penicillins, cephalosporins, etc. by using an entirely different mechanism of attacking bacteria in diseases produced by bacterial infection.

Abbreviations used herin

| | |
|---|---|
| ATP | adenosine triphosphate |
| ADP | adenosine diphosphate |
| AR | androgen receptor |
| CAT | chloramphenicol acyltransferase |
| CBP | CREB binding protein |
| cDNA | complementary DNA |
| DBD | DNA binding domain |
| DMSO | dimethyl sulfoxide |
| DNA | deoxyribonucleic acid |
| DTT | dithiothreitol |
| EDTA | ethylenediaminetetraacetic acid |
| ER | estrogen receptor |
| FPP | farnesyl pyrophosphate |
| GST | glutathione S-transferase |
| HEPES | N-2-Hydroxyethylpiperazine-N′-2-ethanesulfonic acid |
| HSP | heat shock protein |
| IPP | Isopentenyl pyrophosphate |
| kDa | kilodalton(s) |
| LBD | ligand binding domain |
| MR | mineralcorticoid receptor |
| NDP | nucleotide diphosphate |
| NID | nuclear receptor interaction domain |
| NTP | nucleotide triphosphate |
| PAGE | polyacrylamide gel electrophoresis |
| PCR | polymerase chain reaction |
| pI | isoelectric point |
| PPAR | peroxisome proliferator-activated receptor |
| PR | progesterone receptor |
| RAR | retinoid acid receptor |
| RXR | retinoid X receptor |
| SDS | sodium dodecyl sulfate |
| SDS-PAGE | sodium dodecyl sulfate polyacrylamide gel electrophoresis |
| TIF2 | transcription intermediary factor 2 |
| TR | thyroid receptor |
| UPPS | undecaprenyl pyrophosphate synthase |
| VDR | vitamin D receptor |

Amino Acid Abbreviations used herein

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC CUG CUU |
| Arginine | Arg | R | AGA AGG CGA CGC CGG CGU |
| Serine | Ser | S | ACG AGU UCA UCC UCG UCU |

The following examples illustrate various aspects of this invention. These examples do not limit the scope of this invention that is defined by the appended claims.

### EXAMPLE 1

### Expression and Purification of UPPS Prenyltransferase from Streptococcus pneumoniae in Escherichia coli

UPPS from Streptococcus pneumoniae with an additional 20 residues at the amino terminus that include the hexa-histidine tag was over expressed in E. coli, strain BL21(DE3), and purified by NiNTA (nickel nitrilo-tri-acetic acid) column. A plasmid pET28-UPPS was transformed into E. coli BL21 (DE3). For expression E. coli BL21 (pET28-UPPS) was grown at 37° C. in LB medium containing 1% glucose and 50 ug/ml kanamycin to OD600 0.5 and then induced with 1 mM IPTG for 3 hrs. The induced cultures were harvested by centrifugation. The cell paste was suspended in 25 ml Buffer A (10 mM imidazole, 50 mM Na-phosphate, 0.5M NaCl pH7.5) containing 0.1 mg/ml of lysozyme. After incubating on ice 30 min, the cell suspension was put through 4 cycles of sonication, freeze and thaw. The lysate was centrifuged and the supernatant applied to the NiNTA column. The column was washed with 18 ml of Buffer A and 12.5 ml of Buffer B (100 mM imidazole, 50 mM Na-phosphate, 0.5M NaCl pH7.5). The His-tagged UPPS was eluted with 10 ml of Buffer C (500 mM imidazole, 50 mM Na-phosphate, 0.5M NaCl pH7.5). The eluted His-UPPS was dialyzed overnight at 4° C. against 2L of 50 mM Tris-HCl pH 7.5, 0.2M NaCl and 1 mM EDTA. The soluble polypeptide includes 272 amino acid residues with a molecular weight of 29,000. This product was greater than 95% pure by SDS PAGE, has the desired enzymatic activity, and N-terminal amino acid analysis confirmed its identity.

1.A. Measurement of UPPS Activity

Enzymatic activity of UPPS was assayed by measuring the incorporation of (1-14C)IPP (isopentenyl diphosphate) into butanol-soluble materials from the condensations of FPP and (1-14C)IPP using a butanol extraction assay (Shimizu et al. 1998). A typical assay of 100 ul contained 100 mM Tris-HCl, pH7.5, 50 mM KCl, 0.5 mM MgCl2, 0.05% Triton X-100, 0.5 uM FPP, 3.6 uM (1-14C)IPP, and 6 nM purified S. pneumoniae His-UPPS. The reaction was incubated at 25° C. for 25 min and stopped by addition of EDTA to 50 mM. The reaction was extracted with equal volume of 1-butanol and the radioactivity in the butanol phase was measured with a liquid scintillation counter (Shimizu, N., T. Koyama, and K. Ogura. (1998) J. Biol. Chem. 273:19476-19481).

1.B. Ligand Binding to UPPS

It is also possible to define ligand interactions with UPPS in experiments that are not dependent upon enzyme catalyzed turnover of substrates. This type of experiment can be done in a number of ways:

1.B.1. Effects of Ligand Binding upon Enzyme Intrinsic Fluorescence (e.g. of Tryptophan)

Binding of either natural ligands or inhibitors may result in enzyme conformational changes which alter enzyme fluorescence. Using stopped-flow fluorescence equipment, this can be used to define the microscopic rate constants that describe binding. Alternatively, steady-state fluorescence titration methods can yield the overall dissociation constant for binding in the same way that these are accessed through enzyme inhibition experiments.

### EXAMPLE 2

### Crystallization, Structure Determination and Refinement of a Crystal Structure of UPPS from Streptococcus pneumoniae

2.A. Crystallization

Single crystals of native UPPS grew from sitting or hanging drops prepared by mixing 2 μL protein (9.5 mg/ml in 50mM Tris-HCl, pH 7.5, 0.2M NaCl, 1 mM EDTA) with 2 μL of reservoir solution containing either 10-20% ethanol, 0.1M Tris-HCl, pH 8.5 or 4-5% PEG6000, 0.1M HEPES, pH 7.5. The drops were left to equilibrate at room temperature against 500 μL of the reservoir solution. The crystals belong to the orthorhombic crystalline form having a space group P2₁2₁2₁ with unit cell parameters: a=59.6 Å, b=118.0 Å, c=178.2 Å, and two 60 kDa dimers in the asymmetric unit. Some crystals were found to belong to the orthorhombic crystalline form having a space group I2₁2₁2₁ with the similar cell parameters as the primitive cell. In the primitive cell, a pseudo-translation of nearly ½, ½, ½ along the cell edges, results in a diffraction pattern in which the h+k+l=odd are, on the average, markedly weaker than the h+k+l=even reflections resulting in a pseudo body centered lattice. The crystals were quickly transferred to a solution of mother liquor containing 30% xylitol as cryo-protective agent and flash frozen under the cold stream before data collection. The Se-Met substituted protein was expressed in E. coli fed with an amino acid mixture that inhibited the endogenous biosynthesis of methionine and forced the uptake of selenium-labeled methionine from the medium. The protein was crystallized under similar conditions with the exception that the crystallization drops were flushed with argon gas before sealing them in order to prevent oxidation. The crystal structure of UPPS in complex with IPP was determined using native crystals soaked at room temperature for 20-30 minutes in mother liquor containing 2-3 mM IPP and 2 mM MgCl₂. The co-crystals of UPPS in complex with the substrate IPP crystals belong to the orthorhombic crystalline form having the space groups P2₁2₁2₁ and I2₁2₁2₁, similar to the native crystal. The co-crystals of UPPS in complex with the substrate FPP were grown at room temperature from sitting drops prepared by mixing 2 uL of protein solution (˜10 mg/ml, 2 mM FPP, 1,mM MgCl2, 50 mM Tris-HCl pH 7.5 and 200 mM NaCl) with 2uL of reservoir solution containing 100 mM sodium cacodylate, pH 6.4, 120-240 mM sodium acetate. These crystals belong to the monoclinic crystalline form having a space group P2₁ with unit cell dimensions a=58.1 Å, b=44.6 Å, c=115.5 Å, β=98.7°.

2.B. X-ray Diffraction Data Collection

A native UPPS crystal structure was determined by multiwavelength anomalous diffraction, MAD, using the K absorption edge of selenium incorporated into the amino acid methionine. Three diffraction data sets to 2.3 Å resolution were collected at the NSLS's X12C beamline. The wavelengths were determined by analyzing the x-ray fluorescence of the UPPS crystal around the selenium absorption edge. These correspond to the peak (0.9795 Å), the inflection point (0.9791 Å) and at a remote wavelength on the high-energy side of the edge (0.9500 Å). Diffraction intensities from each wavelength were independently integrated, merged and scaled using DENZO/SCALEPACK (Otwinowsky, et al. (1999) Methods in Enzymology Vol. 267). Diffraction data from UPPS in complex with FPP or IPP were collected at the 17ID beamline at the Advanced Photon Source, APS, at Argonne National Laboratory, using 1.000 Å wavelength.

2.C. Structure Determination

A selenium substructure was determined by automatic Patterson map peak search and peak correlation implemented in the program SOLVE (Terwilliger, T. C., and Berendsen, J. (1999) Acta Crystallogr. D55: 849-861). A Fourier map was calculated to 2.7 Å resolution using phases calculated from 15 of the possible 24 Se sites in the asymmetric unit. After solvent modification, this map afforder the determination of the boundaries of the four monomers and tracing of the polypeptide chains. The tracing was used to find the rotation and translation transformations used in 4-fold electron density averaging (CCP4, DM). The improved, averaged map was also used in tracing of the chains.

2.D. Model Building and Refinement

This averaged electron density map was of high quality and afforded the complete tracing of the four molecules using the interactive computer graphics program O (Jones, T. A. et al. (1991) Acta Crystallogr. A47: 110-119). The initial model was refined against diffraction data collected at the remote wavelength by successive rounds of simulated annealing with torsion angle dynamics, positional refinement and restrained B-factor refinement using CNS (A. Brunger et al., Science, 235: 458-460 (1987)) followed by manual intervention. The refinement and manual rebuilding was monitored by the quality of the 2Fo-Fc and Fo-Fc electron density maps and the value of the crystallographic R and R_{free}. At the beginning of the refinement, the four molecules in the asymmetric unit (A, B C, and D) were forced to obey strict non-crystallographic symmetry. This constraint was released as the refinement proceeded.

A final R is 0.20 and the R_{free} is 0.25 for 53,642 reflections to 2.3 Å resolution. The rms deviation from the reference bond lengths and bond angles (Engh & Huber (1991) Acta Crystallogr. A47: 392-400) are 0.01 and 1.4, respectively. The refined model includes residues 17-72, 77-248 in molecule A, 17-72, 79-246 in molecule B, 17-73, 77-248 in molecule C and 18-73, 78-246 in molecule D according to the amino acid sequence SEQ ID NO:1. The N-terminal residues 1-16 were disordered in the four molecules. Also were disordered the residues in the vicinity of the loop formed by amino acids 72-80. A number of conserved amino acids that may form part of the active site (see below) are located in this region. The six residues 247-252 at the C-terminus were also disordered. In the refined model, all the main chain conformations fall in the “allowed” regions of the Ramachandran plot. Molecules A and B form one of the dimers and molecules C and D the second dimer in the asymmetric unit. The Cα-carbon atoms of the two pairs of molecules in each dimer, molecules A and B, and C and D, superimpose with a rms deviation of 1.2 Å and 1.1 Å, respectively, with very large differences at the N-(6.1 Å) and C-termini (1.5 Å), the turn formed by residues 35-41 (1.2 Å), the long helix formed by residues 79-104 (7.1 Å), the short turn formed by residues 115-127 (2.2 Å), and residues 157-171 (1.7 Å) that form an α-helix and a turn. Omitting these residues from the comparison gives a rms of 0.3 Å or both pairs of molecules. The turn formed by residues 35-41 (1.2 Å) in molecule A has no crystal contacts or contacts with the partner in the dimer, however, in molecule B it is making contacts with the sane region in molecule D. The long helix from 79-104 is not making contacts in molecules A or D but in C is contacting residues in the region of B177, and in B is contacting D117 of a symmetry related molecule; this region leads to a disordered loop that may be involved in substrate binding. B115 region is in contact with symmetry related D119-D121, and D115 is contacting symmetry related B85-B88 region. A166 is in contact with symmetry related B240, C166 is in contact with symmetry related D240, but neither B166 nor D166 are making any crystal contacts.

A crystal structure of a UPPS in complex with FPP was determined by molecular replacement with the program package AMoRe (Navaza, J. (1994) Acta Cryst. A50, 157-163) using the native structure as search model and refined as described before. The cross rotation and translation searches were carried using data from 20 Å to 4 Å resolution and a radius of integration of 20 Å. The top two solutions of the cross rotation function corresponding to the tow molecules in the dimer in the asymmetric unit were unambiguously discriminated from the noise peaks. The search for the correct translation for each molecule in the asymmetric unit produced a solution for the tetramer with an R-factor of 0.60 and a correlation coefficient of 0.34 after rigid body refinement in. The crystal structure of the complex with IPP was determined by Fourier methods.

All documents cited herein and patent applications to which priority is claimed are incorporated by reference herein in their entirety. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

TABLE I

Table IA. Atomic coordinates of native UPPS structure

Table IB. Atomic coordinates of active of UPPS in complex with FPP

Table IC. Atomic coordinates of active of UPPS in complex with IPP

TABLE II

Table IIA. Interatomic distances in an active site of the native UPPS

Table IIB. Interatomic distances in an active site of UPPS in complex with FPP

Table IIC. Interatomic distances in an active site of UPPS in complex with IPP

TABLE III

Table IIIA. Interatomic angles in an active site of the native UPPS

Table IIIB. Interatomic angles in an active of UPPS in complex with FPP

Table IIIC. Interatomic angles in an active of UPPS in complex with IPP

Legend:

1. Under the heading ATOM appears a “atom number” (e.g. 1,2,3,4 . . . etc) and the “atom name” (e.g. CA, CB, N, . . . etc) such that to each “atom name” in the coordinate list corresponds an “atom number”.

2. Under the heading RESIDUE appears a three-letter “residue name” (e.g. THR, ASP, etc), a “chain identifier” represented by a capital letter (e.g. A, B, C D, etc) and a “residue number”, such that to each residue (or amino acid) in the amino acid sequence of the particular protein in the structure corresponds a name that identifies it, a number according to its position along the amino acid sequence, and a chain name. The chain name identifies a particular molecule in the crystal structure. For instance, if there are more than one molecule that form the unit that is repeated throughout the crystal lattice, then each unit is identified as molecule A, or molecule B, or molecule C, etc.

3. Under the headings X, Y, or Z appear the Cartesian coordinates of the atoms in the structure.

4. Under the heading OCC appears the “occupancy factor” for each atom. If the entity is present and observed in the structure then occupancy of 1.00 is assigned to it. If the atom is present but not observed, occupancy of 0.00 is assigned to it. Also, factors between 0.00 and 1.00 are also acceptable and represent the degree of confidence in observing that atom a that particular position.

5. Under the heading B appears the “B-factor” or “temperature factor” which can adopt, in principle, any value. It is meant to represent the atomic displacement around that position.

LENGTHY TABLE REFERENCED HERE
US20070020743A1-20070125-T00001
Please refer to the end of the specification for access instructions.

LENGTHY TABLE REFERENCED HERE
US20070020743A1-20070125-T00002
Please refer to the end of the specification for access instructions.

LENGTHY TABLE REFERENCED HERE
US20070020743A1-20070125-T00003
Please refer to the end of the specification for access instructions.

LENGTHY TABLE REFERENCED HERE
US20070020743A1-20070125-T00004
Please refer to the end of the specification for access instructions.

LENGTHY TABLE REFERENCED HERE
US20070020743A1-20070125-T00005
Please refer to the end of the specification for access instructions.

LENGTHY TABLE REFERENCED HERE
US20070020743A1-20070125-T00006
Please refer to the end of the specification for access instructions.

LENGTHY TABLE REFERENCED HERE
US20070020743A1-20070125-T00007
Please refer to the end of the specification for access instructions.

LENGTHY TABLE REFERENCED HERE
US20070020743A1-20070125-T00008
Please refer to the end of the specification for access instructions.

LENGTHY TABLE REFERENCED HERE
US20070020743A1-20070125-T00009
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070020743A1) An electronic copy of the table will also be available from the USPTO upon request and payment of the fee set forth in 37 CFR 1.19(b)(3).

1. A composition comprising a UPPS in crystalline form.

2. The composition according to claim 1 wherein said UPPS is a dimer.

3. The composition of claim 1 wherein said UPPS is a Streptococcus pneumoniae UPPS.

4. The composition according to claim 1 comprising a protein wherein defined by coordinates of Table IA, interatomic distances in Table IIA, or angles of active site residues listed in Table IIIA, in an essentially pure native form or a homolog thereof.

5. A prenyltransferase of claim 4 which is in its native crystalline form.

6. A prenyltransferase according to claim 4 wherein said prenyltransferase has an active site formed by the amino acids Arg247, Gly250, Arg206, Arg200, Ser208, Tyr217, Asp28, Tyr70, Ile26, Phe72, Asn76, Met27, Ala71 as ligands to IPP.

7. A prenyltransferase according to claim 4 wherein said prenyltransferase has an active site formed by the amino acids Asp28-Arg32, Arg79, Met27, His45, Gly48, Met49, Leu52, Ala71, Tyr70, Leu90, Pro91, Phe94, Phe149 as ligands to FPP.

8. A composition comprising the prenyltransferase of claim 4 in complex with the substrate FPP wherein defined by coordinates of Table IB, interatomic distances in Table IIB, or angles of active site residues listed in Table IIIB.

9. A composition comprising the prenyltransferase of claim 4 in complex with the substrate IPP wherein defined by coordinates of Table IC, interatomic distances in Table IIC, or angles of active site residues listed in Table IIICB.

10. A heavy atom derivative of a Streptococcus pneumoniae UPPS crystal wherein the prenyltransferase comprises a protein having the coordinates listed in Tables IA-IC, IIA-IIC, or IIIA-IIIC.

11. A prenyltransferase according to claim 4 wherein said prenyltransferase is characterized by an α+β fold with three layers, αβα, wherein the β-strands form a six-strand parallel β-sheet and three α-helices pack against one face of the sheet and three to four α-helices located on the opposite face.

12. A composition comprising a Streptococcus pneumoniae UPPS in orthorhombic crystalline form having a space group of P2₁2₁2₁.

13. The composition according to claim 12 wherein the crystalline form has lattice constants of a=59.6 Å, b=118.0 Å, c=178.2 Å.

14. The composition according to claim 12 wherein the crystalline form contains two 60 kDa dimers in an asymmetric unit.

15. A composition comprising a Streptococcus pneumoniae UPPS in orthorhombic crystalline form having a space group of I2₁2₁2₁.

16. A composition comprising a co-crystal of Streptococcus pneumoniae UPPS in complex with a substrate IPP in orthorhombic crystalline form having a space group selected from the group consisting of P2₁2₁2₁ and I2₁2₁2₁.

17. A composition comprising a co-crystal of Streptococcus pneumoniae UPPS in complex with a substrate FPP in monoclinic crystalline form having a space group of P2₁.

18. The composition according to claim 17 wherein the crystalline form has lattice constants of a=58.1 Å, b=44.6 Å, c=115.5 Å, β=98.7°.

19. A process for determining a crystal structure form using the structural coordinates of a Streptococcus pneumoniae UPPS crystal or portions thereof, to determine a crystal form of a mutant, homologue, or co-complex of a binding pocket or active site by molecular replacement.

20. A process of identifying an inhibitor compound capable of binding to and inhibiting the enzymatic activity of a Streptococcus pneumoniae UPPS said process comprising:
a) introducing into a suitable computer program information defining an active site conformation of a UPPS molecule comprising a conformation defined by the coordinates and listed in Table IA, IIA, or IIIA wherein said program displays the three-dimensional structure thereof;
b) creating a three dimensional structure of a test compound in said computer program;
c) displaying and superimposing a model of said test compound on a model of said active site;
d) incorporating said test compound in a biological prenyltransferase activity assay for a prenyltransferase characterized by said active site; and
e) determining whether said test compound inhibits enzymatic activity in said assay.

21. A process designing drugs useful for inhibiting UPPS activity using the atomic coordinates of a Streptococcus pneumoniae UPPS crystal to computationally evaluate a chemical entity for associating with a active site of a UPPS enzyme.

22. A method of modifying a test UPPS polypeptide comprising:
a) providing a test UPPS polypeptide sequence having a characteristic that is targeted for modification;
b) aligning the test UPPS polypeptide sequence with at least one reference UPPS polypeptide sequence for which an X-ray structure is available, wherein the at least one reference UPPS polypeptide sequence has a characteristic that is desired for the test UPPS polypeptide;
c) building a three-dimensional model for the test UPPS polypeptide using the three-dimensional coordinates of the X-ray structure(s) of the at least one reference UPPS polypeptide and its sequence alignment with the test UPPS polypeptide sequence;
d) examining the three-dimensional model of the test UPPS polypeptide for a difference in an amino acid residue as compared to the at least one reference polypeptide, wherein the residues are associated with the desired characteristic; and
e) mutating an amino acid residue in the test UPPS polypeptide sequence located at a difference identified in step (d) to a residue associated with the desired characteristic, whereby the test UPPS polypeptide is modified.

23. A process of identifying an inhibitor compound capable of inhibiting the enzymatic activity of a Streptococcus pneumoniae UPPS according to claim 4, said process comprising:
a) carrying out an in vitro assay by introducing said compound in a biological prenyltransferase activity assay containing a prenyltransferase according to claim 4; and
b) determining whether said test compound inhibits the enzymatic activity of the prenyltransferase in said assay.

24. A product of the process of claim 20 which is a peptide, peptidomimetic, or synthetic molecule and is useful for inhibiting a metallo-beta lactamase in treatment of bacterial infections in a mammal.

25. A product according to claim 24 wherein said product is a competitive or non-competitive inhibitor of the Streptococcus pneumoniae prenyltransferase.

26. A process designing drugs useful for inhibiting Streptococcus pneumoniae UPPS comprising using the atomic coordinates of a Streptococcus pneumoniae UPPS crystal or the atomic coordinates of a Streptococcus pneumoniae UPPS in complex with FPP or IPP to computationally evaluate a chemical entity for associating with the active site of a Streptococcus pneumoniae UPPS.

27. The process according to claim 26 comprising the step of using the structure coordinates of Streptococcus pneumoniae UPPS to identify an intermediate in a chemical reaction between a prenyltransferase and a compound that is a substrate or inhibitor of said prenyltransferase.

28. The process according to claim 26 wherein said structure coordinates comprise the coordinates listed in Tables IA-IC, IIA-IIC, or IIIA-IIIC.
